(19) United States
(12) Patent Application Publication
(10) Pub. No.: US 2017/0065022 A1
(43) Pub. Date: Mar. 9, 2017

(54) NOVEL METHOD FOR DESIGNING ATHLETIC FOOTWEAR

(71) Applicant: Courtney Smith, San Jose, CA (US)

(72) Inventor: Courtney Smith, San Jose, CA (US)

(21) Appl. No.: 15/235,098

(22) Filed: Aug. 11, 2016

Related U.S. Application Data
(60) Provisional application No. 62/203,890, filed on Aug. 11, 2015.

(51) Int. Cl. A43B 5/02; A43D 1/02; A61B 5/103; A43B 13/14

(52) U.S. Cl. CPC A43B 5/02; A43B 13/14; A43D 1/02; A61B 5/1038; A61B 2090/066

(57) ABSTRACT

The invention described herein pertains to athletic footwear and in particular to a novel method of designing position-specific athletic footwear. The present invention includes a method of designing athletic footwear comprising collecting data while a set of athletic moves are executed wherein the set of athletic moves are characteristic of a player position for a sport. Based upon the collected data, designing athletic footwear to maximize player position performance.

### PRIORITY

This patent application claims priority to U.S. Provisional Patent Application Ser. No. 62/203,890 entitled “A Novel Method for Designing Athletic Footwear” filed Aug. 11, 2015.

### FIELD

The invention described herein pertains to athletic footwear and in particular to a novel method of designing position-specific athletic footwear.

### BACKGROUND

Conventional athletic footwear and related apparel are designed for specific sports play. In fact, many footwear designers have attempted to improve player performance by designing shoes that seek to maximize performance for each specific sport. For example, Nike Corporation offers many lines of athletic footwear for basketball, football, and track and field in the attempt to maximize an athlete's performance in each of these respective sports.

Although there have been advances in sports performance for athletic footwear, there are still many performance limitations. Many of these performance limitations are due to conventional design objectives which attempt to meet the performance needs of all athletes that play a particular sport, regardless of the various positions, by designing a single shoe for that sport. Most sports, however, have various player positions of which require a different set of repetitive movements. Therefore, a single shoe design will not meet the performance needs for each player position within that sport.

The aforementioned design aim may be effective for athletes which participate in solo sports (e.g., boxing, sprinting, etc.) but may not be as effective for athletes who participate in team sports (e.g., basketball, soccer, football, etc.) which include a plurality of diverse player positions (e.g., football—defensive lineman, offensive wide-out, running back).

Accordingly, there exists a need for a new method of designing athletic footwear. The present disclosure addresses this need.

### BRIEF DESCRIPTION OF THE DRAWINGS

To facilitate understanding, identical reference numerals have been used, where possible, to designate identical elements that are common to the drawings. The drawings are not to scale and the relative dimensions of various elements in the drawings are depicted schematically and not necessarily to scale. The techniques of the present disclosure may readily be understood by considering the following detailed description in conjunction with the accompanying drawings, in which:

FIGS. 1-3 are exemplary prior art footwear for basketball, soccer, and football.

FIG. 4 is an illustration of player positions for the game of basketball.

FIG. 5 is a flowchart of a method for designing athletic footwear consistent with the present disclosure.

FIG. 6A is an exemplary illustration of a system for obtaining data while testing a set of athletic moves.

FIG. 6B is an illustration of the bottom of footwear used while testing a set of athletic moves.

FIG. 7 is an exemplary illustration of exemplary pressure maps obtained while testing of a set of athletic moves.

FIG. 8 is an illustration of athletic footwear designed in accordance with a method consistent with the present disclosure.

### DETAILED DESCRIPTION OF THE PRESENT DISCLOSURE

A detailed description of some embodiments is provided below along with accompanying figures. The detailed description is provided in connection with such embodiments, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For the purpose of clarity, technical material that is known in the technical fields related to some embodiments have not been described in detail to avoid unnecessarily obscuring the description.

The invention described herein pertains to athletic footwear and in particular to a novel method of designing position-specific athletic footwear. The present invention includes a method of designing athletic footwear comprising collecting data while a set of athletic moves are executed wherein the set of athletic moves are characteristic of a player position for a sport. Based upon the collected data, designing athletic footwear to maximize player position performance.

As described herein, an “athletic move” may be defined as any set of bodily movements executed by an athlete to gain (or attempt to gain) a competitive advantage during an athletic competition.

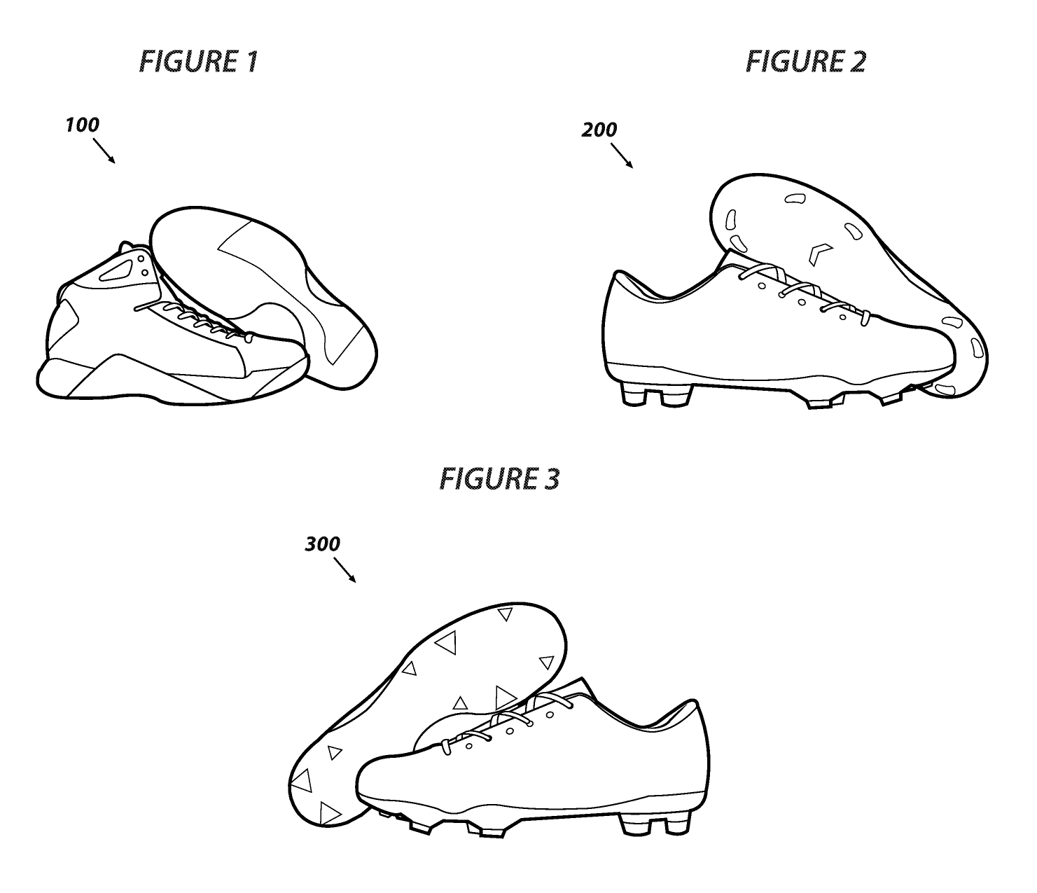

FIGS. 1-3 are exemplary prior art footwear for basketball, soccer, and football. The footwear shown in these figures are designed to meet the performance needs for each respective sport. For example, footwear 100 was designed for basketball players whereas footwear 200 was designed for soccer players. Footwear 300, however, was designed for football players.

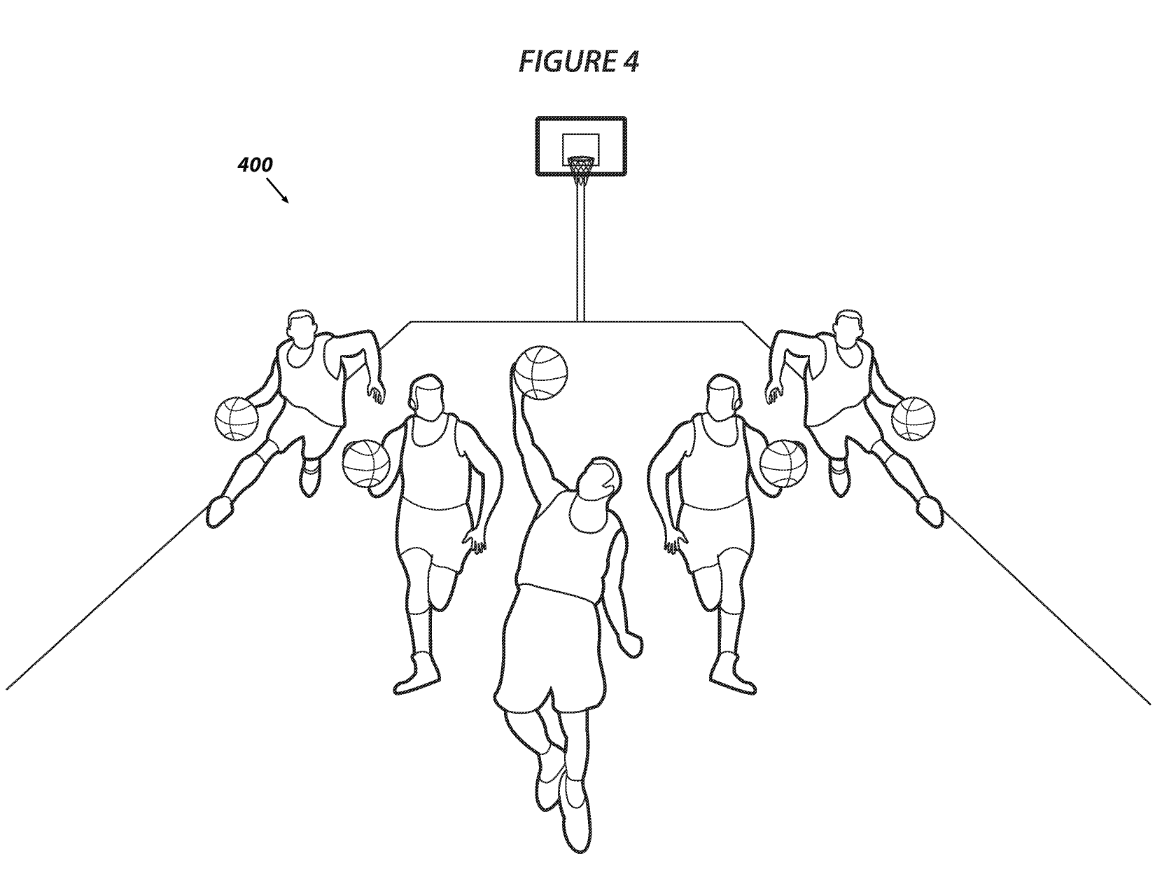

FIG. 4 is an illustration of player positions for the game of basketball. As shown, the sport of basketball includes five player positions—point guard, shooting guard, small forward, power forward, and center. The roles of each player in basketball is unique and therefore requires different performance needs. Accordingly, each player position may benefit from having footwear that is designed specifically for their position.

For instance, a “drop-step” post move is an offensive basketball move commonly employed by a center whereas a “floater” (through the lane) is often employed by point and shooting guards. The footwork required to effect a drop-step post move is different than the footwork required to execute a floater. Accordingly, the stress on the athlete's feet will be different and therefore the performance needs of each athlete's (i.e., center and point/shooting guard) footwear should be different.

It should be understood by one having ordinary skill in the art that the present disclosure is not limited to the game of basketball but that other sports, particular team sports, may be applicable to this invention. For instance, the game of football includes diverse player positions (e.g., a defensive lineman or offensive wide-out). Accordingly, it should be appreciated that the footwear performance needs for each player position.

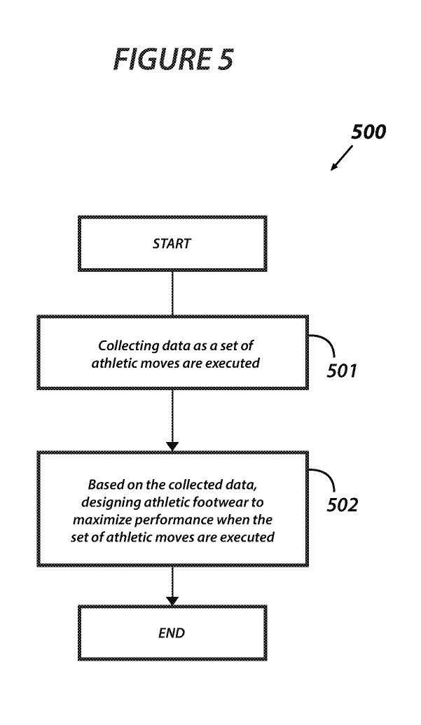

FIG. 5 is a flowchart 500 of a method for designing athletic footwear. The present disclosure is not limited to this method and various modifications may be made thereto without departing from the spirit and scope of the present disclosure.

First, a set of athletic moves characteristic of each particular player position is identified. The set of athletic moves may be identified by any of various methods. For example, one knowledgeable about a particular sport may provide this information (e.g., a coach, player, other team personnel, or enthusiast). Alternatively, advanced computer simulations and other software means may be a source for identifying the set of athletic moves. It should be understood by one having ordinary skill in the art that the manner in which the set of athletic moves is attained does not limit the mode of designing footwear for each player position.

The set of athletic moves may include one or several athletic moves. In some embodiments, the set of athletic moves include only the primary moves associated with the player position. In other embodiments, the set of athletic moves include both primary and secondary moves associated with the player position. The secondary athletic moves may be defined as athletic moves which are attributed to two or more player positions in some implementations.

Moreover, the set of athletic moves may incorporate standard athletic moves associated with a particular sport. For example, in the sport of basketball, players are often required to jump, sprint, and perform lateral movements. As such, the set of athletic moves may incorporate primary, secondary, and/or standard athletic moves depending upon the performance objectives. A basketball point guard is tasked with directing the team's offense. The point guard is a highly athletic position and requires the ability to perform many athletic moves. For example, a few primary athletic moves associated with the point guard position are the following: a floater, euro step, crossover, and jab step and drive/shoot.

Secondary moves may be associated with the point guard position which may also be incorporated within the set of athletic moves associated with this player position. For example, secondary athletic moves for the point guard position may include a V-cut, turnaround jumper, or jump hook. A “lay up” may be considered a standard athletic move since this offensive move is often employed by all player positions in the game of basketball.

Flowchart 500 begins with block 501—collecting data as a set of athletic moves are executed. In some embodiments of the present disclosure, the set of athletic moves are primary moves which are highly associated with a particular player position. However, the set of athletic moves may also include secondary moves. The present disclosure is not limited to any manner of collecting the data so long as it is effective in retrieving feet data and footwear data. For example, electrodes and other sensors may be strategically disposed on a test subject (e.g., the test subject's feet), and their personal effects (e.g., footwear). As will be described in more detail below, the electrical signals detected at the electrodes or other sensors while the athletic moves are executed are sent to a computing device for processing.

A test subject may be an athlete or any other person or apparatus which can effectively execute each athletic move within the set. In some embodiments, the test subject executes each athletic move within the set at near playing conditions such that the data collected therefrom can be used to design footwear which exhibits the best performance for each respective player position.

A plurality of data may be collected. For example, the data collected may include stress data, pressure data, shear data, torsion data, force data, impact data, and the like for both the footwear and the test subject's feet. The data collected during the execution of the athletic moves or set of athletic moves may show a variance with respect to the athletic moves executed. As such, the data collected during the execution of the athletic moves or sets of athletic moves may show a signature for each data type.

More than one test subject may be used for data collection. A plurality of test subjects may be used to generate and collect data for various demographics. For instance, one or more test subjects may be employed to collect data from men, women, girls, and boys of various ages and weight categories to collect a comprehensive data set. The data collected for each demographic may be used to design footwear for each player position per demographic (e.g., for men, women, girls, boys, teens, adults, etc.).

Next, based on said data collection, designing footwear to maximize performance when the set of athletic moves are executed (block 502). The present disclosure makes use of performance data generated during the execution of sets of athletic moves to design footwear that maximizes performance for repetitive or critical athletic moves. Designing footwear, in one embodiment of the present disclosure, includes designing inner soles and outer soles for athletic footwear to boost performance for each player position.

Although footwear design is known in the art, the present disclosure provides a novel approach for which footwear design can be implemented to boost performance over conventional athletic footwear designs. Advantageously, the present disclosure is not limited by sales quotas or other manufacturing constraints. The primary aim of this invention is to boost athletic performance for each player position for each sport. Accordingly, the footwear designed by a method disclosed herein may vary significantly to meet the performance needs as determined by the data collected. For example, footwear designed for a basketball center position may be bulky and restrictive whereas footwear designed for a point guard may be light and flexible. Therefore, the footwear designed for each player position may incorporate different materials to accomplish the performance goals for footwear for each player position.

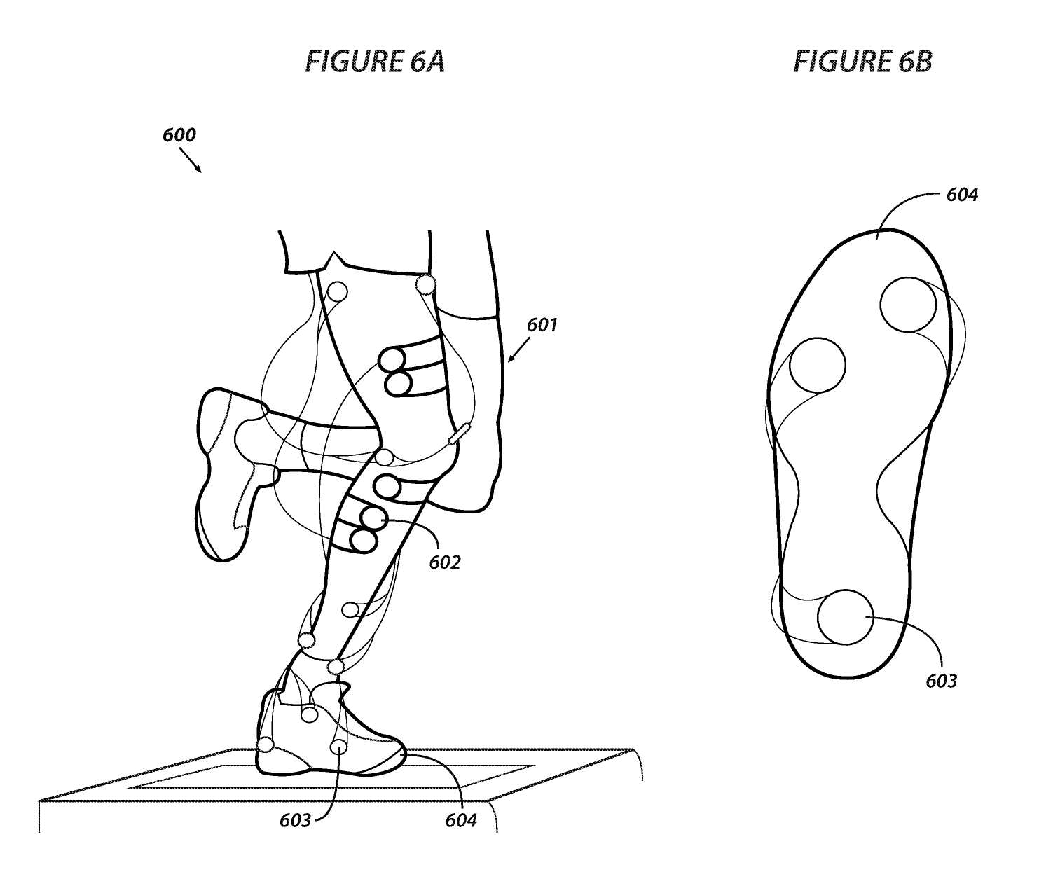

FIG. 6A is an exemplary illustration of a system for obtaining data while testing a set of athletic moves. Shown in the figure is a test subject in a test area 600 equipped with electrodes, sensors, and other data collection devices 602, 603. Additionally, data collection devices 603 are further disposed on the surface of the test subject's footwear 604. The plurality of data collection devices 602 may relay the collected data to a computing device coupled thereto. Advantageously, test area 600 provides a platform for data to be collected for various player positions for various sports.

It should be understood by one having ordinary skill in the art that the present disclosure is not limited to the data collection method illustrated in FIG. 6. The present disclosure is amenable to collect data from wearable devices and circuitry embedded within wearable apparel so long as accurate data can be collected for various athletic movements.

FIG. 6B is an illustration of the bottom of footwear 604 used during the execution of a set of athletic moves. As shown, the bottom of footwear 604 exposes a plurality of sensors 603 that obtain data during test.

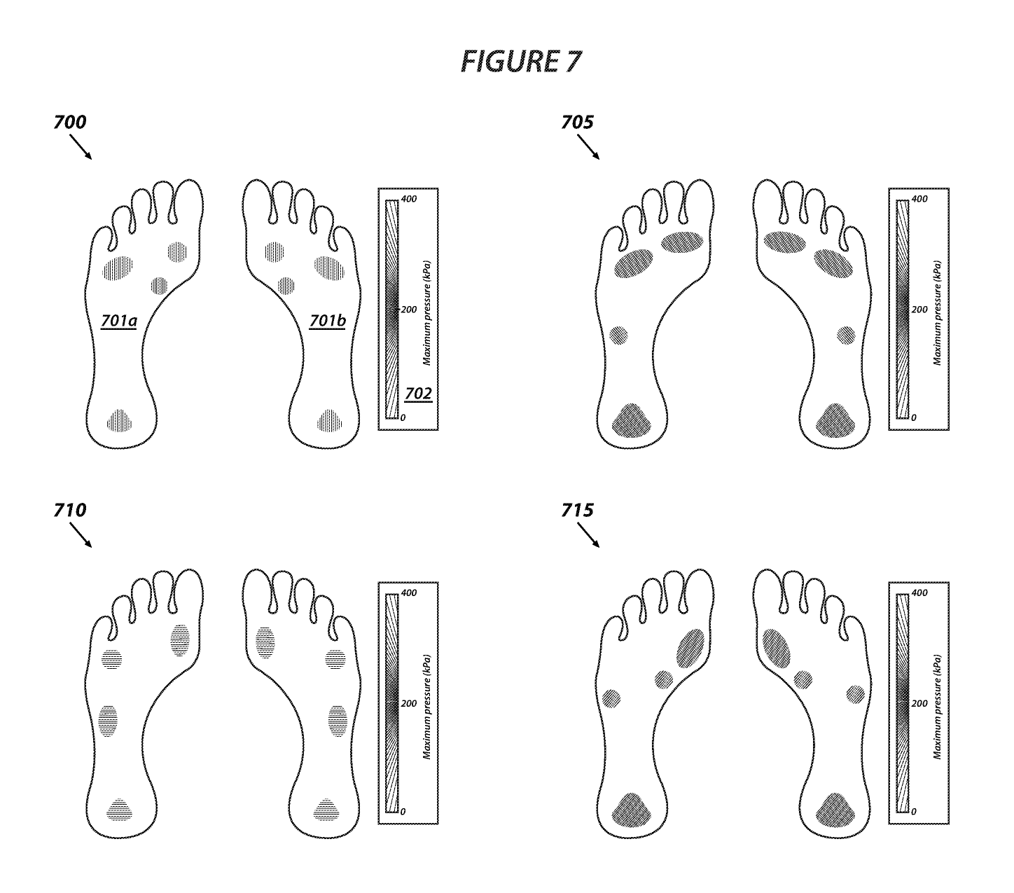

FIG. 7 is an exemplary illustration of exemplary pressure maps obtained while testing a set of athletic moves. Continuing with the point guard position, pressure maps 700, 705, 710, and 715 represent the pressure data obtained during the execution of the identified athletic moves (i.e., floater, euro step, crossover, and jab step and drive/shoot). Pressure map 700 is representative of pressure data obtained from executing a “floater” move. As shown, a plurality of pressure data is “mapped” onto a representation of the test subject's feet 701a, 701b. Key 702 provides a guide to quantifying the pressure data obtained during the execution of the “floater” move. Accordingly, pressure (and other) data may be used to design footwear to maximize the point guard's performance while executing of various athletic moves.

Likewise, pressure maps 705, 710, and 715 display the pressure data obtained during the execution of a euro step, crossover, and jab step. It should be understood by one having ordinary skill in the art that the present disclosure is not limited to utilizing a pressure map to design footwear. A stress map, shear map, torsion map, force map, or impact map may be generated to aid in player-position athletic footwear.

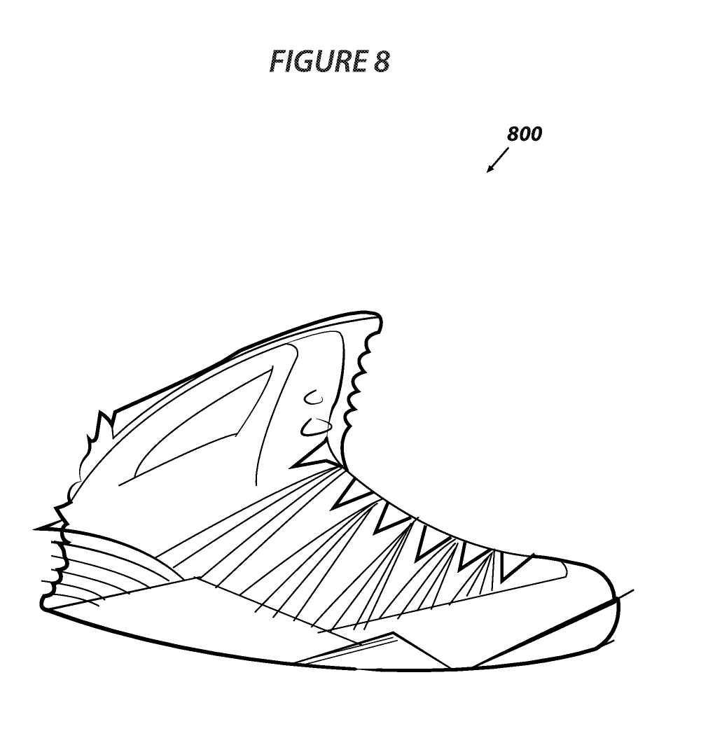

FIG. 8 is an illustration of athletic footwear 800 designed in accordance with a method consistent with the present disclosure. Athletic footwear 800 consistent with the present disclosure was designed for a specific player position. For example, athletic footwear 800 may be designed for a point guard, shooting guard, small forward, power forward, center, wide-out, running back, quarterback, defensive lineman, striker, goalie, sweeper, or any other player position based on the specific data collected from the set of athletic moves tested.

The present disclosure may also be used to design footwear for two or more sports. For example, the set of athletic moves identified and executed may include moves from two or more sports to boost performance for cross training or the like.

Although the present disclosure has been attributed to boosting athletic performance, such disclosure may also be directed to reducing the risk of sports-related injuries. For example, the set of athletic moves tested may incorporate high-risk moves that often lead to injury. For instance, a set of athletic movements may be executed during the test phase that tend to induce critical injuries (e.g., anterior cruciate ligament tear, achilles tear, turf toe, foot fractures, etc.). According to the designer's goals, more or less of these high-risk athletic moves may be executed to generate the requisite data to incorporate into the footwear design.

As such, the present disclosure may be used to design footwear that maximizes performance or reduces the chances of injury during high-risk sports activities. Moreover, footwear may be designed which incorporate both of these objectives.

The preceding Description and accompanying Drawings describe examples of embodiments in some detail to aid understanding. However, the scope of protection may also include equivalents, permutations, and combinations that are not explicitly described herein.

What is claimed is:

1. A method, comprising:
collecting data while a set of athletic moves are executed;
wherein the set of athletic moves are characteristic of a player position for a sport; and
based on the collected data, designing athletic footwear to maximize player position performance.

2. The method of claim 1, further comprising identifying the set of athletic moves that characteristic of the player position for the sport.

3. The method of claim 1, wherein the sport is a team sport.

4. The method of claim 1, wherein the set of athletic moves includes at least one football athletic move, basketball athletic move, or soccer athletic move.

5. The method of claim 1, wherein the data collected includes at least one of stress data, pressure data, shear data, torsion data, force data, or impact data.

6. The method of claim 1, wherein designing athletic footwear consists of designing an inner sole and an outer sole of an athletic footwear.

7. The method of claim 1, wherein the set of athletic moves are performed by a test subject.

8. The method of claim 1, wherein the athletic moves include primary athletic moves and secondary athletic moves.

9. The method of claim 8, wherein the primary athletic moves include athletic moves attributable to one player position.

10. The method of claim 8, wherein the secondary athletic moves include athletic moves attributable to two or more player positions.

11. A player-position sports shoe, comprising:
an integrated design of an inner sole and outer sole of the sports shoe based on data collected during an execution of a set of athletic moves attributable to a player position.

12. The player-position sports shoe of claim 11 is one of a point-guard athletic shoe, a running back athletic shoe, or a pitcher athletic shoe.

13. The player-position sports shoe of claim 11, wherein the data collected includes at least one of stress data, pressure data, shear data, torsion data, force data, or impact data.

14. The player-position sports shoe of claim 11, wherein the sports shoe is designed for a player position of one sport.

15. The player-position sports shoe of claim 11, the sports shoe is designed for a team sport.
